(19) United States
(12) Patent Application Publication
(10) Pub. No.: US 2021/0225052 A1
(43) Pub. Date: Jul. 22, 2021

(54) Smart Display Informational Overlays

(71) Applicant: International Business Machines Corporation, Armonk, NY (US)

(72) Inventors: Mauro Marzorati, Lutz, FL (US); Gary F. Diamanti, Wake Forest, NC (US); Kayla D. White, Wake Forest, NC (US); Craig M. Trim, Ventura, CA (US)

(21) Appl. No.: 16/749,590

(22) Filed: Jan. 22, 2020

(51) Int. Cl. G06T 11/60; G16H 20/60; G06N 20/00; G06K 9/00

(52) U.S. Cl. CPC G06T 11/60; G06K 9/00718; G06N 20/00; G16H 20/60

(57) ABSTRACT

A method, system, and computer program product are provided for selectively overlaying information by analyzing a received display image to identify products in the display image and by applying an artificial intelligence machine learning analysis to the identified products to identify product content information for each identified product and to evaluate the product content information against viewer health criteria to generate a predicted health-related interaction for the viewer which is used to generate a display overlay or augmentation for the display image for providing feedback to the viewer by displaying on the display screen the display overlay or augmentation with the display image.

### BACKGROUND OF THE INVENTION

With the increasing amount of advertisements and other visual content provided over a multitude of platforms, consumers are constantly bombarded with product information that cannot readily be processed by the consumer to determine if a product is suitable. For example, food products advertised on television may contain ingredients that may not be appropriate to the age or health restrictions for a particular viewer if that viewer does not know the makeup of the product. There are health and fitness tools, such as the NxtNutrio app, that can identify chemicals, preservatives, and additives contained in foods, these tools typically require the user to enter their allergen (e.g., GMO, MSG, corn, etc.), and then use a barcode scan as an input interface to scan the barcode of any product to identify the nutritional risks for the user. However, such tools are of limited use in terms of assisting the consumers to ability to receive personalized information identifying problematic interactions with products included in advertisements or other visual content. For example, the barcode databases of existing health and fitness tools typically recognize most major brand names, they do not include every product. In addition, they only work with products that have barcodes. There are also existing systems for overlaying advertising and purchasing information which uses on-line and streaming media in combination with a product panel for selecting product(s) to be purchased, customized, or to receive additional dynamic information (such as real-time feeds of the current price of the stock, RSS, tickers, marquees etc.), static information (such as hyperlinks to other web sites). As a result, the existing solutions for helping consumers make informed decisions about the suitability of products for the individual consumer's needs are deficient at a practical and/or operational level.

### SUMMARY

Broadly speaking, selected embodiments of the present disclosure provide a personalized information overlay device, system, method, and apparatus for receiving and processing video/image information to generate and display personalized health-related information which augments and/or overlays the displayed video/image. In selected embodiments, the personalized health-related information is generated by dynamically identifying and classifying different objects or entities in the video/image with a cognitive learning system to identify product content information (e.g., ingredients, calories, nutrition, allergens, etc.) for each identified entity for evaluation against personalized user health criteria or information. Once generated, the personalized health relevant information may be displayed to the viewer as a real-time overlay display or augmentation over the video/image. In selected embodiments, the disclosed personalized information overlay device, system, method, and apparatus are configured to analyze images and identify products and content in the products to form a history, and to apply artificial intelligence machine learning techniques to the history based on user-specific relevancy criteria (e.g., health or dietary requirements, such as allergens or dietary restrictions) to establish a predicted relevancy. As new images are received and analyzed to identify products (e.g., food items) and related product contents (e.g., food ingredients), the artificial intelligence machine learning techniques are applied to the new image to predict a new image relevancy which may be evaluated against a predetermined relevancy threshold (e.g., a calorie count or limit) when determining whether to overlay or augment the new image with real-time information related to the predicted new image relevancy.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention, as defined solely by the claims, will become apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1A illustrates an example display screen image showing a soft drink being consumed with a first example display panel of personalized health-related information which overlays the displayed image in accordance with selected embodiments of the present disclosure.

FIG. 1B illustrates an example display screen image showing milk being poured into a glass with a second example display panel of personalized health-related information which overlays the displayed image in accordance with selected embodiments of the present disclosure.

FIG. 1C illustrates a side-by-side display of a screen image in which identified products are identified with personalized health-related information overlays for two different consumers in accordance with selected embodiments of the present disclosure.

FIG. 2 diagrammatically depicts a system having a personalized information overlay device connected in a network environment to a computing system wherein a smart display overlay engine identifies product content information for each identified entity in an image and evaluates the product content information against personalized user health criteria to generate a real-time overlay display of personalized health-related information in accordance with selected embodiments of the present disclosure;

FIG. 3 is a block diagram of a processor and components of an information handling system such as those shown in FIG. 2; and

FIG. 4 illustrates a simplified flow chart showing the logic for aiding consumer choices by generating a display overlay of personalized health-related information in accordance with selected embodiments of the present disclosure.

### DETAILED DESCRIPTION

The present invention may be a system, a method, and/or a computer program product. In addition, selected aspects of the present invention may take the form of an entirely hardware embodiment, an entirely software embodiment (including firmware, resident software, micro-code, etc.) or an embodiment combining software and/or hardware aspects that may all generally be referred to herein as a “circuit,” “module” or “system.” Furthermore, aspects of the present invention may take the form of computer program product embodied in a computer readable storage medium (or media) having computer readable program instructions thereon for causing a processor to carry out aspects of the present invention. Thus embodied, the disclosed system, a method, and/or a computer program product is operative to improve the functionality and operation of a smart display cognitive information overlay computing system by efficiently processing image information to generate and display personalized health-related information in a display overlay or augmentation.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a dynamic or static random access memory (RAM), a read-only memory (ROM), an erasable programmable read-only memory (EPROM or Flash memory), a magnetic storage device, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server or cluster of servers. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments described herein provide a personalized information overlay device, system, method, and apparatus which permit a consumer or viewer to quickly and efficiently identify and display personalized health-related information related to products displayed on a video screen or smart device. The personalized information overlay device combines video processing and artificial intelligence techniques to generate and display personalized health-related information which augments and/or overlays the displayed video/image with configurable video overlay content based on user preferences or health data. The personalized information overlay device may detect objects displayed in received video/images, retrieve associated ingredients or contents of each identified object from a health corpus of medical or dietary information, model the health-related interactions between the consumer and the associated ingredients or contents using a machine learning model and user-specified health criteria, and present personalized health-related guidance output based on the model by displaying personalized health-related information which augments and/or overlays the displayed video/image. As disclosed, the personalized information overlay device may be connected to a video output device to perform visual recognition of objects displayed on a screen by applying a binary classifier model to identify or match detected images on the display with entity labels (e.g., milk container, soda bottle and brand, cereal box brands, etc.) and then applying a predictive model (a linear regression model) to determine if the identified product's content (e.g., ingredients and/or allergens) are allowed or not for the viewer. To train the predictive model, content from a health data corpus (e.g., FDA corpus, medical journals, etc.) that is directed to medical conditions/afflictions or dietary requirements is used to train a machine learning model, such as a linear regression model, to process the ingredients list for each product identified on the screen for specified health criteria (e.g., known allergens or counterindications within the product and/or the user's known afflictions, dietary restrictions, and/or medical conditions) and to generate personalized health-related information in response to the ingredients list for a product matching with or correlating to the specified health criteria. In some embodiments, the personalized information overlay device may correlate the dietary data (e.g., the sugar content or calorie counts of an identified product on the screen) against a threshold health measure (e.g., the viewer's glucose level or caloric budget) of the observer of the video images. Once generated, the personalized health-related information may be displayed to the viewer as a real-time overlay display or augmentation over the video/image with any desired visual overlay or augmentation.

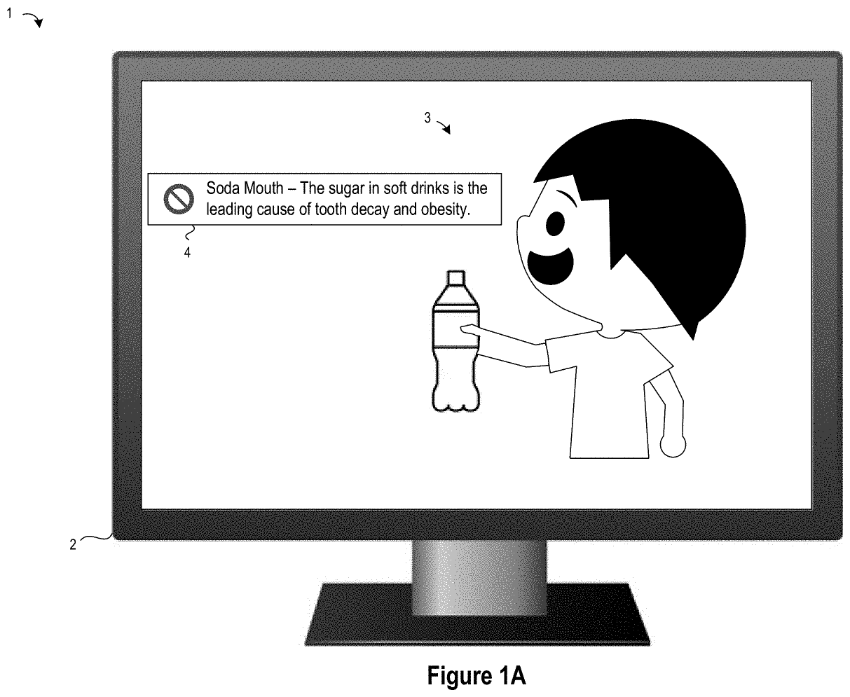

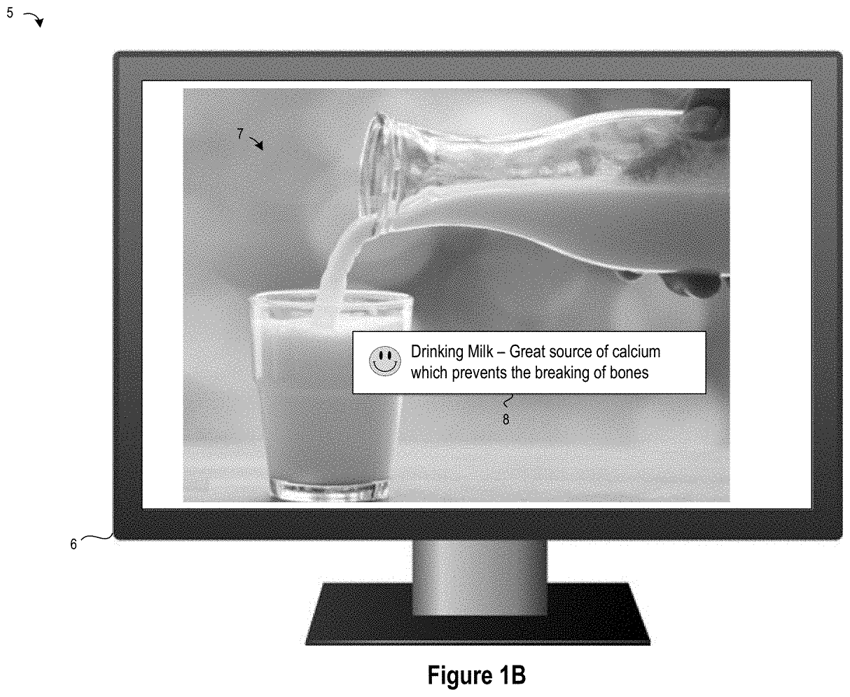

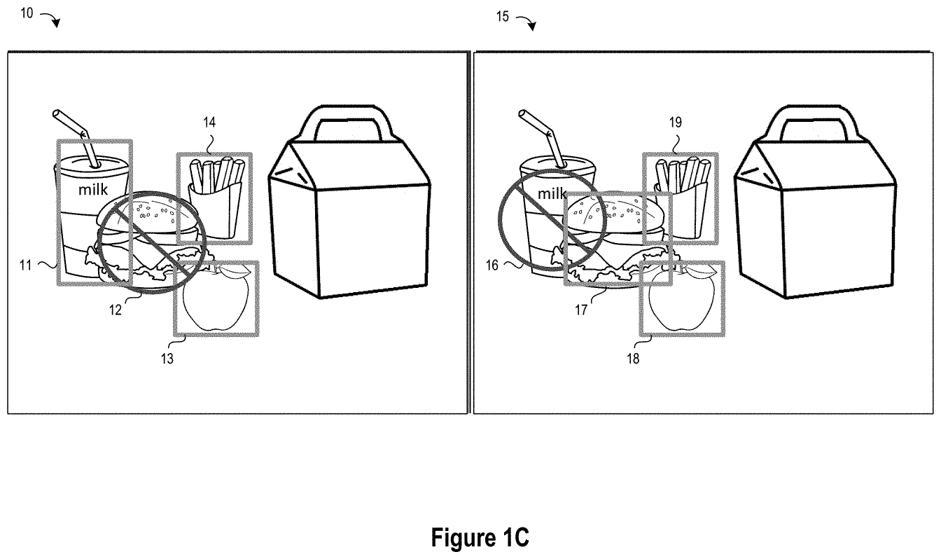

As will be appreciated, the personalized information overlay device may include a variety of different components, such as sensors that obtain data regarding the user's health status or condition, input/output mechanisms for receiving input from and/or providing input to the user, processing units and/or other components for generating the model and/or mapping the model to various input/output mechanisms, and so on. These and other embodiments are discussed below with reference to FIGS. 1-4. However, those skilled in the art will readily appreciate that the detailed description given herein with respect to these Figures is for explanatory purposes only and should not be construed as limiting.

To provide additional details for an improved understanding of selected embodiments of the present disclosure, reference is now made to FIG. 1A provides an illustration 1 of example display screen 2 depicting an image 3 showing a boy consuming a soft drink from a bottle. In the displayed image 3, an example display panel 4 is overlaid to display personalized health-related information about the soft drink product displayed in the image 3. To this end, a personalized information overlay device may be configured to process the image 3 that is included in a commercial advertisement (e.g., as part of a television program or video) by using one or more machine learning, natural language processing (NLP), and/or artificial intelligence (AI) processing techniques to classify or identify a product match for the displayed soft drink, to locate ingredients for the matching product, and to generate the overlay panel 4 which visually identifies a negative or problematic interaction with the health of the viewer. In this example, the problematic interaction is identified with a negative “no” sign with appropriate coloring and/or a written statement (e.g., “Soda Mouth—The sugar in soft drinks is the leading cause of tooth decay and obesity.”).

To provide additional details for an improved understanding of selected embodiments of the present disclosure, reference is now made to FIG. 1B provides an illustration 5 of example display screen 6 depicting an image 7 showing milk being poured into a glass. In the displayed image 7, an example display panel 8 is overlaid to display personalized health-related information about the milk product displayed in the image 7. To this end, a personalized information overlay device may be configured to process the image 7 that is included in a commercial advertisement by using one or more machine learning, natural language processing (NLP), and/or artificial intelligence (AI) processing techniques to classify or identify a product match for the displayed milk product, to locate ingredients for the matching product, and to generate the overlay panel 8 which visually identifies a positive or non-problematic interaction with the health of the viewer. In this example, the non-problematic interaction is identified with a positive “happy face” sign with appropriate coloring and/or a written statement (e.g., “Drinking Milk—Great source of calcium which prevents the breaking of bones.”).

To provide additional details for an improved understanding of selected embodiments of the present disclosure, reference is now made to FIG. 1C provides a side-by-side illustration of two display screen images 10, 15 for two different consumers. In each of the first and second displayed images 10, 15 there is depicted an advertisement image of a lunch meal which includes the meal container or box, along with the meal components, including chicken nuggets, milk drink, fries, and fruit. However, the personalized health-related information that included in the display overlay for each image is adjusted relative to the sensitivity or health criteria for the user viewing each image.

For example, the health criteria of a first viewer of the first displayed image 10 may be used to generate a first set of display panels 11-14 that is overlaid on top of the meal components to display personalized health-related information about the meal components based on the health criteria of the first viewer. In this example, a viewer of the first displayed image 10 may have gluten sensitivity, in which case the personalized information overlay device may be configured to process the image 10 using one or more models to identify the chicken nuggets, milk drink, fries, and fruit products in the image 10, to locate ingredients for each identified matching product, and to generate the first set of display panels 11-14 which visually identifies the positive and/or negative interactions with the health of the first viewer. In this example where the first viewer is sensitive to glutens contained in the chicken nuggets, the positive interactions are identified with green overlay boxes (e.g., a first green overlay box 11 around the milk drink, a second green overlay box 14 around the fries, and a third green overlay box 13 around the fruit), while negative interactions are identified with red overlay boxes (e.g., a first red overlay box 12 around the chicken nuggets).

However, the health criteria of a second viewer of the displayed image 15 may be used to generate a second set of display panels 16-19 that is overlaid on top of the meal components to display personalized health-related information about the meal components based on the health criteria of the second viewer. In this example, a viewer of the first displayed image 10 may be lactose intolerant, in which case the personalized information overlay device may be configured to process the image 15 using one or more models to identify the chicken nuggets, milk drink, fries, and fruit products in the image 15, to locate ingredients for each identified matching product, and to generate the second set of display panels 16-19 which visually identifies the positive and/or negative interactions with the health of the second viewer. In this example where the second viewer has sensitivity to lactose contained in the milk, the positive interactions are identified with green overlay boxes (e.g., a first green overlay box 17 around the chicken nuggets, a second green overlay box 18 around the fruit, and a third green overlay box 19 around the fries), while negative interactions are identified with red overlay boxes (e.g., a first red overlay box 16 around the milk drink).

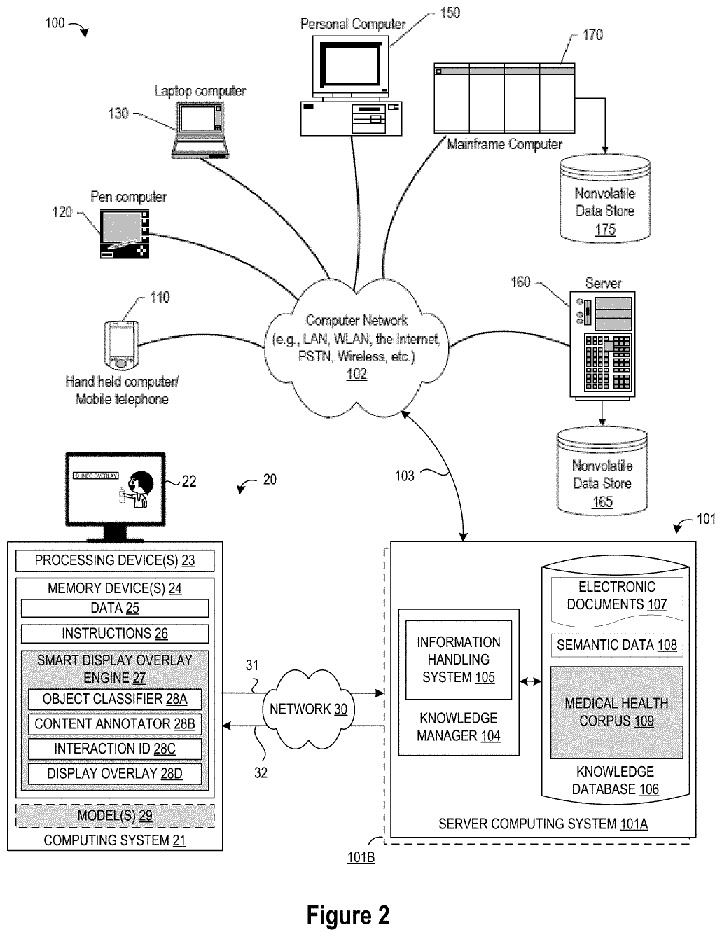

To provide additional details for an improved understanding of selected embodiments of the present disclosure, reference is now made to FIG. 2 which schematically depicts a system diagram 100 of one illustrative embodiment of a personalized information overlay device 20 which includes a computing system 21 and visual output device 22 connected in a network environment 30 to a server computing system 101. The depicted personalized information overlay device 20 uses a smart display overlay engine 27 to provide identify product content information for each identified entity in an image and to evaluate the product content information against personalized user health criteria to generate a real-time overlay display 22 of personalized health-related information. In the depicted example, the visual output device 22 may be embodied as a display screen on a smart TV, tablet, or other motion video display, such as a wearable device (e.g., watch or smart glasses). As depicted, the visual output device 22 displays an image, such as the image 3 shown in FIG. 1A.

The visual image displayed by the information captured by the visual output device 22 may be processed in whole or in part with the computing system 21 which is illustrated as including one or more processing devices 23 and one or more memory devices 24 which are operatively connected together with other computing device elements generally known in the art, including buses, storage devices, communication interfaces, and the like. For example, the processing device(s) 23 may be used to process visual image data received at the computing system 21 for storage in the memory device(s) 24 which stores data 25 and instructions 26. In accordance with selected embodiments of the present disclosure, the stored data 25 and instructions 26 may embody a smart display overlay engine 27 which is configured to process video/image data and provide overlay content images or augmentations on the user's video screen 22 or smart devices based on user preferences and/or criteria (e.g., health or dietary conditions) by using one or more machine learning models 29 which are trained on a medical health corpus 109 that is focused on the end user's medical afflictions or dietary requirements.

To this end, the smart display overlay engine 27 may include a first classifier module 28A for dynamically identifying and classifying different objects or entities Ei in the captured visual information (e.g., video image) on the display screen 22 as the user or agent is viewing the video content shown on the display screen 22. In selected embodiments, the first classifier module 28A may employ any suitable image classification service or object detection algorithm, such as Convolutional Neural Networks (CNN), binary classifier, or the like, to identify the objects or entities that are included in the captured visual information while the user/agent is viewing the video content. For example, an object classifier module 28A may be used to monitor the image frames in the captured visual content, and then identify different entities E (E1, E2, . . . En) in the image frames. As will be appreciated, the smart display overlay engine 27 analyzes the captured visual information to identify objects or entities using a model 29 that may be trained by a human subject matter expert who annotates images with labels by looking at the image and assigning a label to the image. The model 29 then gets volumes of additional images to teach it the intended domains for model usage or prediction. When the model 29 is then given a new, previously unseen image, it will predict what the new image is. In this way, the image classification module 28A may be applied to ingest or break down the input display image pixel-by-pixel into a pattern which is associated with a matching label, such as cat or mouse. In selected embodiments, the object classification function may be performed in real time on the video image being displayed, but may also retrieve object classification information that was previously identified (e.g., when the same video was played) and stored in memory 24, thereby providing the ability to “forecast” the appearance of known products on the screen based on processing of the same sequence of video images in the past.

To further assist with object/entity identification, the smart display overlay engine 27 may also include a second content annotator module 28B for generating and/or accessing a product content list (e.g., ingredients and/or allergens) for each identified entity/product appearing on the display screen 22. As will be appreciated, the second content annotator module 28B may be trained on corpus content (e.g., food labels, FDA data, etc.) where a human subject matter expert annotates or assigns labels or categories to training data images or objects in a “supervised learning” process. In selected embodiments, the second content annotator module 28B may employ any suitable classification algorithm or model 29, such as a binary classifier model, or the like, to detect and match each identified entity/product with a corresponding product content list. To this end, the second content annotator module 28B may retrieve a product content list from a medical health corpus 109 that is ingested from multiple public domain sources, such as food ingredient labels, FDA corpus, medical journals, and the like. For example, the corpus 109 may include ingredients listed in product packaging, such as a ravioli product which includes “eggs” and “milk and wheat” as listed ingredients. In addition, the corpus 109 may include prioritized food grouping information for promoting healthy outcomes, such as ordered priority of food groups in the “healthy eating pyramid” guide or the “healthy eating plate” guide.

The smart display overlay engine 27 may also include a third interaction identification module 28C for measuring or detecting the health interactions between the viewer and the content of each identified product/entity identified in the display screen 22. In selected embodiments, the third interaction identification module 28C may employ any suitable predictive model, including but not limited to a linear and logic regression model, to predict if an identified or matched product/entity is healthy for the viewer based on the corpus it was trained on. To personalize the predictive model 29 to the viewer of the displayed content, the model 29 may ingest user-specific health data about the health and/or dietary condition of the viewer, such as the viewer's age, food allergies, medical conditions, religious or dietary restrictions, amount of food or calories consumed, etc. The predictive model 29 will then predict what type of information should be included as an overlay or augmentation for the displayed product. For example, the third interaction identification module 28C may process the product contents for all products identified on the display screen 22 for negative health interactions (e.g., known allergens or other health or dietary counterindications for the viewer) and/or for positive health interactions (e.g., healthy food choices, recommended daily servings of food groups, etc.) that are tailored or specific to the viewer.

To enable personalized user feedback of positive and/or negative health interactions between the viewer and the content of each identified product/entity identified in the display screen 22, the smart display overlay engine 27 may also include a fourth display overlay module 28D to provide personalized feedback information about the health interactions in the form of personalized health-related information which augments and/or overlays the displayed video/image 22. In selected embodiments, the fourth display overlay module 28D may generate a visual overlay for the area of the screen containing any product that is detrimental to the viewer, where the visual overlay may be a colored square, circle or other polygon. In addition or in the alternative, the generated visual overlay may include iconography and/or coloring to indicate or identify the allergen or condition/affliction in an identified product which should not be used or consumed by the viewer. In addition or in the alternative, the generated visual overlay may identify the caloric content of an identified product, alone or as a percentage of the viewer's available caloric budget for the day. In selected embodiments, the computation of the viewer's available caloric budget may be altered or modified by taking into account reservations for near-term future consumptions (e.g., dinner party scheduled on the user's calendar).

As indicated with the dashed lines around the model(s) 29 block, the model(s) may be located in whole or in part in the computing system 21, but may alternatively be located in the server computing system 101 which is connected to exchange data 31, 32 over the network 30 with the personalized information overlay device 20. In such embodiments, the server computing system 101 may include one or more system pipelines 101A, 101B, each of which includes a knowledge manager computing device 104 (comprising one or more processors and one or more memories, and potentially any other computing device elements generally known in the art including buses, storage devices, communication interfaces, and the like) for processing information data 31 received from the personalized information overlay device 20, as well as information data 103 received over the network 102 from one or more users at computing devices (e.g., 110, 120, 130). Over the network(s) 30, 102, the computing devices communicate with each other and with other devices or components via one or more wired and/or wireless data communication links, where each communication link may comprise one or more of wires, routers, switches, transmitters, receivers, or the like. In this networked arrangement, the computing systems 21, 101 and networks 30, 102 may be used with components, systems, sub-systems, and/or devices other than those that are depicted herein.

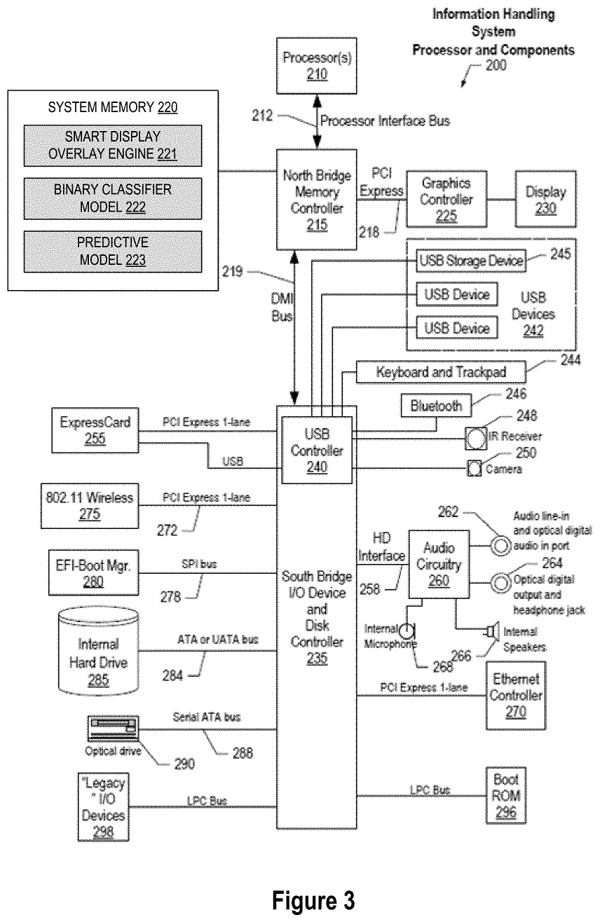

In the server computing system 101, the knowledge manager 104 may be configured with an information handling system 105 to receive inputs from various sources. For example, knowledge manager 104 may receive input from the network 102, one or more knowledge bases or corpora 106 of electronic documents 107, semantic data 108, or other data, content users, and other possible sources of input. In selected embodiments, the knowledge base 106 may include structured, semi-structured, and/or unstructured content in a plurality of documents that are contained in one or more large knowledge databases or corpora. For example, the knowledge base 106 may include a medical health corpus 109 of medical or dietary information which is used to train the model(s) 29. In addition, the server computing system 101 may be connected to communicate with different types of information handling systems which range from small handheld devices, such as handheld computer/mobile telephone 110 to large mainframe systems, such as mainframe computer 170. Examples of handheld computer 110 include personal digital assistants (PDAs), personal entertainment devices, such as MP3 players, portable televisions, and compact disc players. Other examples of information handling systems include pen or tablet computer 120, laptop or notebook computer 130, personal computer system 150, and server 160. As shown, the various information handling systems can be networked together using computer network 102. Types of computer network 102 that can be used to interconnect the various information handling systems include Local Area Networks (LANs), Wireless Local Area Networks (WLANs), the Internet, the Public Switched Telephone Network (PSTN), other wireless networks, and any other network topology that can be used to interconnect the information handling systems. Many of the information handling systems include nonvolatile data stores, such as hard drives and/or nonvolatile memory. Some of the information handling systems may use separate nonvolatile data stores (e.g., server 160 utilizes nonvolatile data store 165, and mainframe computer 170 utilizes nonvolatile data store 175). The nonvolatile data store can be a component that is external to the various information handling systems or can be internal to one of the information handling systems. An illustrative example of an information handling system showing an exemplary processor and various components commonly accessed by the processor is shown in FIG. 3.

To provide additional details for an improved understanding of selected embodiments of the present disclosure, reference is now made to FIG. 3 which illustrates an information handling system 200, more particularly, a processor and common components, which is a simplified example of a computer system capable of performing the computing operations described herein. Information handling system 200 includes one or more processors 210 coupled to processor interface bus 212. Processor interface bus 212 connects processors 210 to Northbridge 215, which is also known as the Memory Controller Hub (MCH). Northbridge 215 connects to system memory 220 and provides a means for processor(s) 210 to access the system memory. In the system memory 220, a variety of programs may be stored in one or more memory devices, including a smart display overlay engine module 221 which may be invoked for processing video/image data shown on a viewer's display screen to dynamically identify and classify different products and associated product content information for purposes of evaluation against user-specific health criteria in order to generate and display personalized health-related information which augments and/or overlays the displayed video/image. In addition or in the alternative, the system memory 220 may include one or more artificial intelligence (AI) machine learning (ML) models, such as a binary classifier model 222 which is trained to detect/match certain images of food/ingredients and/or a predictive model 223 which is trained to determine if the identified food products are allowed for viewer consumption (or not) based on interactions between the product ingredients and specified health criteria for the viewer. Graphics controller 225 also connects to Northbridge 215. In one embodiment, PCI Express bus 218 connects Northbridge 215 to graphics controller 225. Graphics controller 225 connects to display device 230, such as a computer monitor.

Northbridge 215 and Southbridge 235 connect to each other using bus 219. In one embodiment, the bus is a Direct Media Interface (DMI) bus that transfers data at high speeds in each direction between Northbridge 215 and Southbridge 235. In another embodiment, a Peripheral Component Interconnect (PCI) bus connects the Northbridge and the Southbridge. Southbridge 235, also known as the I/O Controller Hub (ICH) is a chip that generally implements capabilities that operate at slower speeds than the capabilities provided by the Northbridge. Southbridge 235 typically provides various busses used to connect various components. These busses include, for example, PCI and PCI Express busses, an ISA bus, a System Management Bus (SMBus or SMB), and/or a Low Pin Count (LPC) bus. The LPC bus often connects low-bandwidth devices, such as boot ROM 296 and “legacy” I/O devices (using a “super I/O” chip). The “legacy” I/O devices (298) can include, for example, serial and parallel ports, keyboard, mouse, and/or a floppy disk controller. Other components often included in Southbridge 235 include a Direct Memory Access (DMA) controller, a Programmable Interrupt Controller (PIC), and a storage device controller, which connects Southbridge 235 to nonvolatile storage device 285, such as a hard disk drive, using bus 284.

ExpressCard 255 is a slot that connects hot-pluggable devices to the information handling system. ExpressCard 255 supports both PCI Express and USB connectivity as it connects to Southbridge 235 using both the Universal Serial Bus (USB) the PCI Express bus. Southbridge 235 includes USB Controller 240 that provides USB connectivity to devices that connect to the USB. These devices include webcam (camera) 250, infrared (IR) receiver 248, keyboard and trackpad 244, and Bluetooth device 246, which provides for wireless personal area networks (PANs). USB Controller 240 also provides USB connectivity to other miscellaneous USB connected devices 242, such as a mouse, removable nonvolatile storage device 245, modems, network cards, ISDN connectors, fax, printers, USB hubs, and many other types of USB connected devices. While removable nonvolatile storage device 245 is shown as a USB-connected device, removable nonvolatile storage device 245 could be connected using a different interface, such as a Firewire interface, etc.

Wireless Local Area Network (LAN) device 275 connects to Southbridge 235 via the PCI or PCI Express bus 272. LAN device 275 typically implements one of the IEEE 802.11 standards for over-the-air modulation techniques to wireless communicate between information handling system 200 and another computer system or device. Extensible Firmware Interface (EFI) manager 280 connects to Southbridge 235 via Serial Peripheral Interface (SPI) bus 278 and is used to interface between an operating system and platform firmware. Optical storage device 290 connects to Southbridge 235 using Serial ATA (SATA) bus 288. Serial ATA adapters and devices communicate over a high-speed serial link. The Serial ATA bus also connects Southbridge 235 to other forms of storage devices, such as hard disk drives. Audio circuitry 260, such as a sound card, connects to Southbridge 235 via bus 258. Audio circuitry 260 also provides functionality such as audio line-in and optical digital audio in port 262, optical digital output and headphone jack 264, internal speakers 266, and internal microphone 268. Ethernet controller 270 connects to Southbridge 235 using a bus, such as the PCI or PCI Express bus. Ethernet controller 270 connects information handling system 200 to a computer network, such as a Local Area Network (LAN), the Internet, and other public and private computer networks.

While FIG. 3 shows one information handling system, an information handling system may take many forms, some of which are shown in FIG. 2. For example, an information handling system may take the form of a desktop, server, portable, laptop, notebook, or other form factor computer or data processing system. In addition, an information handling system may take other form factors such as a personal digital assistant (PDA), a gaming device, ATM machine, a portable telephone device, a communication device or other devices that include a processor and memory. In addition, an information handling system need not necessarily embody the north bridge/south bridge controller architecture, as it will be appreciated that other architectures may also be employed.

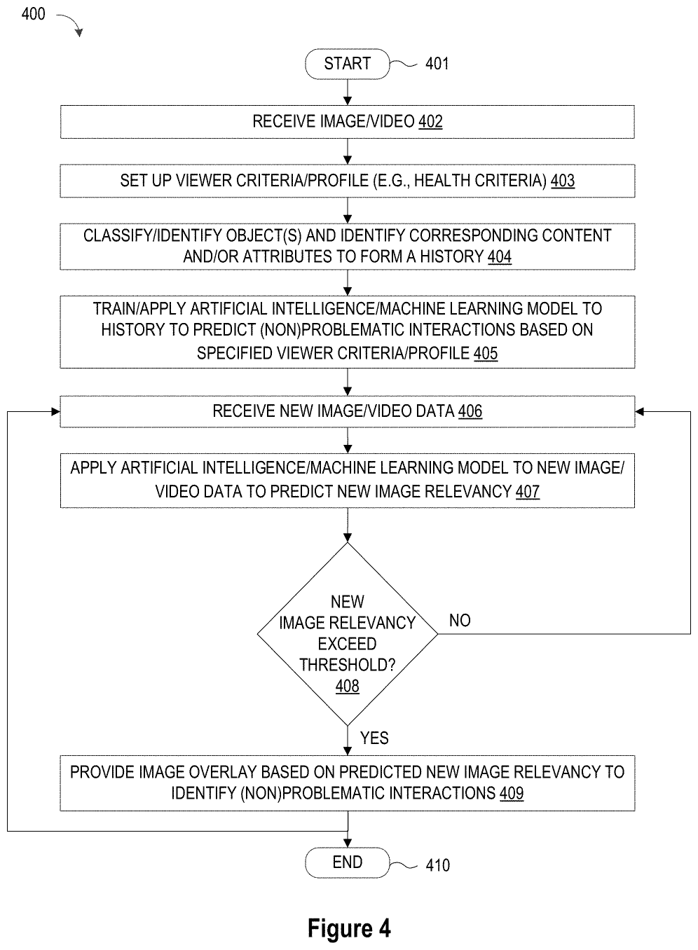

To provide additional details for an improved understanding of selected embodiments of the present disclosure, reference is now made to FIG. 4 which depicts a simplified flow chart 400 showing the logic for aiding consumer choices by generating a display overlay of personalized health-related information. The processing shown in FIG. 4 may be performed by a cognitive system, such as the first computing system 21, server computing system 101, or other question answering system.

FIG. 4 processing commences at 401, such as when a personalized information overlay device is connected or attached to a video or image playback device, such as a display screen on a smart TV, tablet, or other motion video display, such as a wearable device (e.g., watch or smart glasses) and the user activates a video or image playback.

At step 402, the image or video is received, such as when the personalized information overlay device receives the image or video data that is displayed on the playback device. For example, an adhoc wireless or Bluetooth network may be established between the display screen of a wearable device (e.g., watch or smart glasses) and the personalized information overlay device so that the image or video can be received and processed at the personalized information overlay device. At this time, an additional communication link may be established with a remote server computer system which hosts one or more artificial intelligence machine learning models and/or a medical/health corpus. As will be appreciated, any suitable network connection may be used to connect to the remote server computer system, including, but not limited to Intranet, Internet, Wireless communication network, Wired communication network, Satellite communication network, etc.

At step 403, viewer criteria and/or profile information is set up with one or more user configuration or specialization data files. In selected embodiments, the viewer criteria/profile may specify the health/medical/dietary criteria for the viewer which will be used to specify the types of health-related image overlay content or augmentation based on the viewer's individual health/medical/dietary conditions. The viewer criteria may also specify the age, dietary goals, allergies, and/or eating disorders of the viewer, as well as applicable healthy eating priorities, such as low cholesterol, lean protein, low starch vegetables, whole grains, healthy fats, and fruit.

At step 404, one or more objects in the received image/video are identified and/or classified, and corresponding content and/or attributes for each identified object is identified to form a history. In selected embodiments, a first computing system (e.g., computing system 21) may process captured image/video data from the display screen with a smart display overlay engine to classify or identify objects or entities in the image/video data. For example, an image classification algorithm, such as a binary classifier, may be executed on the personalized information overlay device to identify objects as products (e.g., food products, drinks, sandwiches, snacks, fruit, cereal, vegetables, milk containers, soda bottles and brands, cereal box brands, etc.) in the image/video data while the viewer is watching the display screen. In addition, the first computing system (e.g., computing system 21) may process captured image/video data using any suitable classifier model to identify corresponding content and/or attributes for each identified object, such as by retrieving product content information (e.g., ingredients, calories, nutrition, allergens, etc.) for each identified product/object.

In building up a history of identified objects and corresponding content/parameters with the processing at step 404, the personalized information overlay device can retrieve the history from memory to effectively forecast the appearance of known products on the display screen when the same image/video data is subsequently played back. In other embodiments, the personalized information overlay device is trained to recognize products in real time as the products appear on the display screen. In addition, the personalized information overlay device may be trained to identify or process the corresponding content/parameters (e.g., ingredients and/or allergen lists) for several specific products appearing on the display screen.

At step 405, machine learning, natural language processing (NLP), and/or artificial intelligence (AI) processing techniques are applied, alone or in combination, to train one or more models to automatically predict problematic and/or nonproblematic interactions from the history based on specified viewer criteria/profile data. In selected embodiments, a server computing system (e.g., computing system 101) and/or first computing system 21 may employ artificial intelligence processing techniques using one or more machine learning models which are trained with the specified viewer criteria/profile and the history of identified product/objects E (E1, E2, . . . En) and corresponding content/attributes to predict or determine if an identified product's content (e.g., ingredients, allergens, caloric counts, etc.) is allowed or not for the viewer. In selected embodiments, content from a health data corpus (e.g., food ingredient labels, FDA corpus, medical journals, etc.) that is directed to medical conditions/afflictions or dietary requirements is used to train the machine learning model, such as a linear regression model, to process the ingredients list for each product identified on the screen for specified health criteria (e.g., known allergens or counterindications within the product and/or the user's known afflictions, dietary restrictions, and/or medical conditions) and to generate personalized health-related information in response to the ingredients list for a product matching with or correlating to the specified health criteria. Over multiple training periods or iterations/epochs, the machine learning model is able to adapt to the viewer's health criteria and help the viewer evaluate the health interactions from products identified in the image/video shown on the display screen.

At step 406, a new image or video is received at the playback device for display on the display screen. As will be appreciated, the new image may be received in real time, such as when a new video frame is received from the video content being displayed on the playback device. Alternatively, the new image may be received when a video program or image displayed on the playback device.

At step 407, one or more machine learning, natural language processing (NLP), and/or artificial intelligence (AI) processing techniques are applied, alone or in combination, to the new image/video data to predict a new relevancy value with respect to problematic and/or nonproblematic interactions based on specified viewer criteria/profile data. In selected embodiments, a server computing system (e.g., computing system 101) and/or first computing system 21 may employ artificial intelligence processing techniques using one or more machine learning models (e.g., a binary classifier model and/or predictive model) which are trained with the specified viewer criteria/profile and the history of identified product/objects E (E1, E2, . . . En) and corresponding content/attributes to predict or determine if any products identified in the new image have corresponding product content (e.g., ingredients, allergens, caloric counts, etc.) that is allowed or not allowed for the viewer.

At step 408, the new image relevancy value is compared to a predetermined threshold. If the new image relevancy does not exceed the predetermined threshold (negative outcome to detection step 408), then no further action is taken, and the methodology returns to step 406 to await detection of a new image. However, if the new image relevancy does exceed the predetermined threshold (affirmative outcome to detection step 408), then the method proceeds to overlay information on the new image related to the predicted new image relevancy.

At step 409, feedback is provided to the user when an identified product/object Ei is displayed which has problematic (or non-problematic) interactions with the viewer. In selected embodiments, the feedback is provided to the viewer by generating a visual overlay for the area of the display screen containing any product that is detrimental to the viewer's health or medical condition, where the visual overlay may be a square, circle or other polygon that is colored a first color (e.g., red) to indicate a problematic or negative interaction, and is colored a second color (e.g., green) to indicate a non-problematic or positive interaction. In addition or in the alternative, the generated visual overlay may include iconography and/or text to indicate or identify the allergen or condition/affliction in an identified product which should not be used or consumed by the viewer, such as a text overlay stating that “The sugar in soft drinks is the leading cause of tooth decay.” In addition or in the alternative, the generated visual overlay may identify the caloric content of an identified product, alone or as a percentage of the viewer's available caloric budget for the day. In addition or in the alternative, the generated visual overlay may include a visual augmentation to indicate that an identified product should not be used or consumed by the viewer. An example visual augmentation could include a facial overlay of decayed teeth on a person drinking a sugary soft drink to visually indicate the negative effects that sugar has on the human body over time. Other visual augmentations and/or overlay content for specific products or ingredients may be built up and/or crowd-sourced by third parties.

In addition to providing the user feedback at step 409, the process is iteratively repeated to continue processing newly received image/video data (step 406), and this iterative process continues until the process ends at step 410. As a result, a method and system provide configurable smart display informational overlays on a user's video screen or smart devices which are personalized to the user based on user preferences and/or health criteria. To provide a personalized overlay of health-related information for display with the received image/video, one or more machine learning models are trained to identify, from received video or images, displayed products and associated product attributes or contents using data from a corpus that is focused on the end user's medical afflictions or dietary requirements, as well as product contents or ingredients for any products identified in the received video/images. With the disclosed embodiments, there are numerous advantages obtained. For example, the ability to use machine learning models trained with public domain content to identify health information related to displayed products can help educate users and improve health/disease/dietary awareness. The techniques disclosed herein can be tailored for specific audiences and users, such as educating children, elderly individuals, or users who are new to their condition/ailment about how everyday foods/product can affect them. There are additional education benefits of explaining to individual viewers the health benefits/risks over time (e.g., sugar effects on tooth decay or obesity). The user-specified health criteria can also be specified to provide parental controls which block children viewers from seeing unhealthy food advertisements/choices when the visual overlays are used to cover or block images of unhealthy items. In other embodiments, the personalized information overlay device may be paired with a home assistant or grocery delivery service to buy suggested products to meet the viewer's specific health needs. In such embodiments, the paired service may have certain product options or choices switched out or substituted if the viewer's health criteria indicate that some products (e.g., cake) are not healthy for the viewer.

By now, it will be appreciated that there is disclosed herein a system, method, apparatus, and computer program product for selectively overlaying display information over a display image. As disclosed, an information handling system comprising a processor and a memory receives a display image for viewing on a display screen by a viewer. In selected embodiments, the display image is received by capturing a video image from a video being displayed on the display screen. At the information handling system, the display image is analyzed to identify one or more products in the display image. In selected embodiments, the display image is analyzed by employing a binary classifier model to identify the one or more products from the video image while the viewer is watching the video. In addition, an artificial intelligence (AI) machine learning analysis is applied to the identified one or more products to identify product content information for each identified product and to evaluate the product content information against viewer health criteria to generate a predicted health-related interaction for the viewer. In selected embodiments, the artificial intelligence (AI) machine learning analysis is applied by deploying a linear regression learning model to evaluate the product content information against viewer health criteria to generate a predicted health-related interaction for the viewer. Based on the predicted health-related interaction for the viewer, the information handling system generates a display overlay or augmentation for the display image which is provided as feedback to the viewer by displaying on the display screen the display overlay or augmentation with the display image. In selected embodiments, the artificial intelligence (AI) machine learning analysis is applied by applying a machine learning (ML) model to a history of product content information for products displayed to the viewer on the display screen to compute a predicted relevancy based on the viewer health criteria. Upon receiving real-time a new image sent to the display screen, the machine learning (ML) model may applied against the new image to predict a new image relevancy, and in response to determining that the new image relevancy exceeds a predetermined threshold, the information handling system overlays real-time information related to the predicted new image relevancy over the new image. In such embodiments, the viewer health criteria may include a plurality of food ingredients, and the real-time information identifies a plurality of substances (e.g., an allergen) in a food product exceeding a threshold quantity. In other embodiments, the viewer health criteria may include dietary restrictions for the viewer, and the real-time information identifies a plurality of substances (e.g., calories) in a food product exceeding a threshold quantity (e.g., a calorie count). As disclosed herein, the predetermined threshold may be based on a relationship with other ingredients in the new image or consumed by the viewer that day.

While particular embodiments of the present invention have been shown and described, it will be obvious to those skilled in the art that, based upon the teachings herein, changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A computer-implemented method for selectively overlaying display information over a display image, the method comprising:
receiving, by an information handling system comprising a processor and a memory, the display image for viewing on a display screen by a viewer;
analyzing, by the information handling system, the display image to identify one or more products in the display image;
applying an artificial intelligence (AI) machine learning analysis to the identified one or more products to identify product content information for each identified product and to evaluate the product content information against viewer health criteria to generate a predicted health-related interaction for the viewer;
generating, by the information handling system, a display overlay or augmentation for the display image which is based on the predicted health-related interaction for the viewer; and
providing feedback, by the information handling system, to the viewer by displaying on the display screen the display overlay or augmentation with the display image.

2. The computer-implemented method of claim 1, where receiving the display image comprises capturing a video image from a video being displayed on the display screen.

3. The computer-implemented method of claim 2, where analyzing the display image comprises employing a binary classifier model to identify the one or more products from the video image while the viewer is watching the video.

4. The computer-implemented method of claim 3, where applying the artificial intelligence (AI) machine learning analysis comprises deploying a linear regression learning model to evaluate the product content information against viewer health criteria to generate a predicted health-related interaction for the viewer.

5. The computer-implemented method of claim 1, where applying the artificial intelligence (AI) machine learning analysis comprises:
applying a machine learning (ML) model to a history of product content information for products displayed to the viewer on the display screen to compute a predicted relevancy based on the viewer health criteria;
receiving real-time a new image sent to the display screen;
applying the machine learning (ML) model against the new image to predict a new image relevancy; and
responsive to determining that the new image relevancy exceeds a predetermined threshold, overlaying real-time information related to the predicted new image relevancy over the new image.

6. The computer-implemented method of claim 5, where the viewer health criteria comprises a plurality of food ingredients and the real-time information identifies a plurality of substances in a food product exceeding a threshold quantity.

7. The computer-implemented method of claim 6, where the plurality substances comprises an allergen for the viewer.

8. The computer-implemented method of claim 6, where the threshold quantity is a calorie count.

9. The computer-implemented method of claim 5, where the predetermined threshold is a based on a relationship with other ingredients in the new image or consumed by the viewer that day.

10. An information handling system comprising:
one or more processors;
a memory coupled to at least one of the processors;
a set of instructions stored in the memory and executed by at least one of the processors to selectively overlay display information over a display image, wherein the set of instructions are executable to perform actions of:
receiving, by the system, the display image for viewing on a display screen by a viewer;
analyzing, by the system, the display image to identify one or more products in the display image;
applying an artificial intelligence (AI) machine learning analysis to the identified one or more products to identify product content information for each identified product and to evaluate the product content information against viewer health criteria to generate a predicted health-related interaction for the viewer;
generating, by the system, a display overlay or augmentation for the display image which is based on the predicted health-related interaction for the viewer; and
providing feedback, by the system, to the viewer by displaying on the display screen the display overlay or augmentation with the display image.

11. The information handling system of claim 10, wherein the set of instructions are executable to analyze the display image by employing a binary classifier model to identify from the display image to identify the one or more products while the viewer is watching the display image.

12. The information handling system of claim 10, wherein the set of instructions are executable to apply the artificial intelligence (AI) machine learning analysis by deploying a linear regression learning model to evaluate the product content information against viewer health criteria to generate a predicted health-related interaction for the viewer.

13. The information handling system of claim 10, wherein the set of instructions are executable to applying the artificial intelligence (AI) machine learning analysis by:
applying a machine learning (ML) model to a history of product content information for products displayed to the viewer on the display screen to compute a predicted relevancy based on the viewer health criteria;
receiving real-time a new image sent to the display screen;
applying the machine learning (ML) model against the new image to predict a new image relevancy; and
responsive to determining that the new image relevancy exceeds a predetermined threshold, overlaying real-time information related to the predicted new image relevancy over the new image.

14. The information handling system of claim 13, where the viewer health criteria comprises a plurality of food ingredients, where the real-time information identifies a plurality of substances in a food product exceeding a threshold quantity, and where the threshold quantity is a calorie count.

15. The information handling system of claim 14, where the viewer health criteria comprises a plurality of food ingredients, where the predetermined threshold is a based on a relationship with other ingredients in the new image or consumed by the viewer that day.

16. The information handling system of claim 13, where the viewer health criteria comprises a plurality of food ingredients, where the real-time information identifies a plurality of substances in a food product exceeding a threshold quantity, and where the plurality substances comprises an allergen for the viewer.

17. A computer program product stored in a computer readable storage medium, comprising computer instructions that, when executed by an information handling system, causes the system to selectively overlay display information over a display image by:
receiving, by the system, a display image from a video being displayed on a display screen for viewing by a viewer;
analyzing, by the system, the display image to identify one or more products in the display image;
applying an artificial intelligence (AI) machine learning analysis to the identified one or more products to identify product content information for each identified product and to evaluate the product content information against viewer health criteria to generate a predicted health-related interaction for the viewer;
generating, by the system, a display overlay or augmentation for the display image which is based on the predicted health-related interaction for the viewer; and
providing feedback, by the system, to the viewer by displaying on the display screen the display overlay or augmentation with the display image.

18. The computer program product of claim 17, further comprising computer instructions that, when executed by the system, causes the system to analyze the display image by employing a binary classifier model to identify the one or more products from the display image while the viewer is watching the video.

19. The computer program product of claim 18, further comprising computer instructions that, when executed by the system, causes the system to apply the artificial intelligence (AI) machine learning analysis by deploying a linear regression learning model to evaluate the product content information against viewer health criteria to generate a predicted health-related interaction for the viewer.

20. The computer program product of claim 17, further comprising computer instructions that, when executed by the system, causes the system to apply the artificial intelligence (AI) machine learning analysis by:
applying a machine learning (ML) model to a history of product content information for products displayed to the viewer on the display screen to compute a predicted relevancy based on the viewer health criteria;
receiving real-time a new image sent to the display screen;
applying the machine learning (ML) model against the new image to predict a new image relevancy; and
responsive to determining that the new image relevancy exceeds a predetermined threshold, overlaying real-time information related to the predicted new image relevancy over the new image.
